(19) United States
(12) Patent Application Publication
(10) Pub. No.: US 2016/0095830 A1
(43) Pub. Date: Apr. 7, 2016

(54) CO-ADMINISTRATION OF INTRAVENOUS IBUPROFEN AND ACETAMINOPHEN FOR TREATMENT OF PAIN

(71) Applicant: Cumberland Pharmaceuticals Inc., Nashville, TN (US)

(72) Inventors: Leo PAVLIV, Cary, NC (US); Amy Dix ROCK, Nashville, TN (US)

(73) Assignee: Cumberland Pharmaceuticals Inc., Nashville, TN (US)

(21) Appl. No.: 14/875,054

(22) Filed: Oct. 5, 2015

Related U.S. Application Data
(60) Provisional application No. 62/060,198, filed on Oct. 6, 2014.

(51) Int. Cl. A61K 31/192; A61K 9/00; A61K 31/167

(52) U.S. Cl. CPC A61K 31/192; A61K 31/167; A61K 9/0019

(57) ABSTRACT

An effective dose of intravenous ibuprofen administered co-adminstered with intravenous acetaminophen in surgery patients is a safe and effective way to reduce both pain and the need for opioid analgesics. In preferred embodiments, the administration of intravenous ibuprofen and intravenous acetaminophen starts no later than at approximately the completion of wound closure.

### FIELD OF THE INVENTION

Provided are methods for treating pain and/or reducing the need for narcotic analgesics and/or adverse events and/or antiemetic medication post surgery by intravenously administering a pharmaceutical composition comprising co-administration of therapeutically effective amounts of intravenous ibuprofen and acetaminophen.

### BACKGROUND OF THE INVENTION

2-(4-isobutylphenyl) propionic acid, whose International Nonproprietary Name is ibuprofen, is a well-known anti-inflammatory drug having a molecular weight of 206.28 and the following chemical structure:

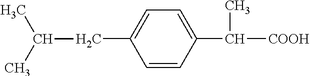

(Merck Index 12th ed., n4925, page 839). Originally patented in the 1960's, ibuprofen is now marketed generically, as well as under the tradenames of Motrin®, Advil®, and Nuprin® for the treatment of pain, inflammation, and fever. The U.S. Food and Drug Administration recently approved a new formulation of ibuprofen for intravenous administration to be marketed under the trade name Caldolor®.

Ibuprofen is readily available as the racemic mixture ((RS)-Ibuprofen) of the two enantiomers, (R)-Ibuprofen and (S)-Ibuprofen. Even though the (S) enantiomer is the biologically active form, most preparations contain the racemic mixture since the (R) enantiomer is converted to the active (S) form in-vivo. For simplicity, hereinafter the term “ibuprofen” will be used to indicate any one of the (R) enantiomer, the (S) enantiomer, or the racemate.

Ibuprofen is currently approved for use as oral treatment for minimal to moderate pain from arthritis, surgery, sunburn, menstruation, and fever. Like aspirin and other drugs in the NSAID family, ibuprofen is believed to reduce the inflammatory response by inhibiting the formation of prostaglandins. Several studies have demonstrated the success of oral or rectal ibuprofen in the reduction of fever and the subjective symptoms associated with it.

Ibuprofen is also available as an investigational intravenous preparation and has been studied in Phase 2 and Phase 3 placebo controlled trials of patients with fever and severe sepsis. In these studies, intravenous ibuprofen reduced fever and pulse rate and lessened lactic acidosis in patients with sepsis. These studies also demonstrated that ibuprofen administered intravenously was safe as determined by detailed evaluation of renal function, gastrointestinal bleeding, transfusion requirements, and other serious adverse events (SAEs). Additional clinical studies have evaluated the safety and pharmacokinetics of intravenous ibuprofen formulations given to healthy adult volunteers.

Although ibuprofen has many advantages over other analgesics such as aspirin and acetaminophen, it is very poorly soluble in water. Thus, certain dosage forms of ibuprofen, especially injectable liquids, have been difficult to develop. Several U.S. patents have addressed this problem.

For example, U.S. Pat. No. 4,309,421 appears to describe water-soluble complexes of ibuprofen and phospholipids suitable for parenteral administration. U.S. Pat. Nos. 4,859,704 and 4,861,797 appear to describe the synthesis of alkali metal salts of ibuprofen for preparing a liquid ibuprofen formulation.

Other U.S. patents appear to address this problem by preparing an ibuprofen salt with a basic amino acid as the active pharmaceutical ingredient and then solubilizing the salt to produce a liquid dosage form.

For example, U.S. Pat. No. 5,200,558 appears to describe enhanced analgesic effects of S (+) ibuprofen as salts of L and D amino acids, including arginine, in various dosage forms, including as an injectable solution. U.S. Pat. No. 4,279,926 appears to describe the use of basic amino acid salts of propionic acids for relieving pain and treating inflammatory conditions. Similarly, U.S. Pat. No. 5,463,117 appears to describe the preparation of salts of ibuprofen with basic amino acids. Finally, U.S. Pat. No. 6,005,005 appears to describe a liquid composition for oral use containing ibuprofen and arginine.

U.S. Pat. No. 6,727,286 B2 describes, among other things, a pharmaceutical composition comprising an aqueous solution of arginine and ibuprofen, wherein the molar ratio of arginine to ibuprofen is less than 1:1, as well as a method of making the same. That patent also provides a method of treating a condition chosen from pain, inflammation, fever, and/or other conditions alleviated by ibuprofen comprising administering a pharmaceutical composition comprising an aqueous solution of arginine and ibuprofen, wherein the molar ratio of arginine to ibuprofen is less than 1:1. The entire contents of U.S. Pat. No. 6,727,286 B2 are hereby incorporated herein by reference.

The U.S. Food and Drug Administration recently approved a new formulation of ibuprofen for intravenous administration to be marketed under the trade name Caldolor® by Cumberland Pharmaceuticals, Inc. Caldolor® contains the active ingredient ibuprofen. As described on the labeling for Caldolor®, “each 1 mL of solution contains 100 mg of ibuprofen in Water for Injection, USP. The product also contains 78 mg/mL arginine at a molar ratio of 0.92:1 arginine:ibuprofen. The solution pH is about 7.4.” Caldolor® is sterile and is intended for intravenous administration only.

Caldolor® possesses antiinflammatory, analgesic, and antipyretic activity. As such, Caldolor® is indicated in adults for the management of mild to moderate pain and the management of moderate to severe pain as an adjunct to opioid analgesics. 400 mg to 800 mg of Caldolor® is administered intravenously every 6 hours as necessary to treat pain. Caldolor® is also indicated for the reduction of fever in adults. 400 mg of Caldolor® is administered intravenously, followed by 400 mg every 4 to 6 hours or 100-200 mg every 4 hours as necessary to treat fever.

Physicians have options in terms of pain and fever control, but each seems to come with a trade-off. It would be highly desirable to provide a new development in the management of pain and fever which improves patient care.

Prior publications report that analgesic potency may be improved while reducing undesirable effects by combining an opioid with an NSAID or an analgesic such as acetylsalicylic acid or acetaminophen, in such a way as to obtain a synergistic analgesic effect allowing for a reduction in the total dose of both the NSAID and analgesic. For example, U.S. Pat. No. 4,569,937, issued to Baker et al. on Feb. 11, 1986, describes a combination of oxycodone with ibuprofen in a ratio of oxycodone/ibuprofen from 1:6 to about 1:400. U.S. Pat. No. 4,690,927, issued to Voss et al. on Sep. 1, 1987, describes a combination of the NSAID diclofenac and codeine in a weight ratio of diclofenac to codeine of about 1:1 to about 3:1. U.S. Pat. No. 5,190,947, issued to Riess et al. on Mar. 2, 1993, describes a diclofenac-codeine salt ([2-[2,6-dichlorophenyl)-amino]-phenyl]-acetic acid). U.S. Pat. No. 4,844,907, issued to Elger et al. on Jul. 4, 1989, describes a multiphase tablet combining a narcotic analgesic phase and an NSAID phase in separate layers. U.S. Pat. No. 4,587,252, issued to Arnold et al. on May 6, 1986, describes a process for treating pain using a combination of hydrocodone and ibuprofen.

### OBJECTS AND SUMMARY OF THE INVENTION

It is another object of the invention to provide a treatment for surgical pain.

It is another object of the invention to provide a method of treating human patients for surgical pain who are being treated with opioid analgesics in a manner which reduces opioid side effects.

It is another object of the invention to provide a method of treating human patients for surgical pain who are being treated with opioid analgesics which is opioid sparing.

In accordance with the above objects and others, the present invention is directed in part to the co-administration of intravenous ibuprofen and acetaminophen to a human patient(s) either prior to, during, or after surgery.

The invention is further directed to a method for reducing pain in a human patient(s) undergoing surgical procedures, comprising intravenously ibuprofen pre-surgically to the patient in an amount effective to significantly reduce post-operative pain in the patients, administering acetaminophen no later than at the time of surgical wound closure, and thereafter co-administering therapeutically effective doses of intravenous ibuprofen and acetaminophen about every 4 to about every 6 hours post-surgery, for at least about 8 doses, and in certain embodiments for up to about 120 hours (5 days) post-surgery. In certain preferred embodiments, the administration of ibuprofen/acetaminophen in this manner reduces pain without the need for the administration of an opioid analgesic and/or delay the time to need for an opioid analgesic and/or reduces the amount of opioid analgesic administered to the patient post-surgery.

The invention is also directed in part to the administration of intravenous ibuprofen/acetaminophen to a patient(s) who is/are being treated with an opioid analgesic(s) for pain, and thereby providing an opioid-sparing effect, enabling the reduction of the dose of opioid to the patient. The administration of intravenous ibuprofen/acetaminophen in such situations further provides a reduction in side effects associated with the administration of opioid analgesics. Further, the administration of intravenous ibuprofen/acetaminophen may reduce pain scores (e.g., VAS scores) in patients who are concurrently administered opioid analgesics, as compared to patients receiving opioid analgesics alone.

In further embodiments, the invention is directed in part to a method of treating human patient(s) for pain associated with a surgical procedure, comprising administering an effective dose of intravenous ibuprofen prior to surgery and an effective dose of acetaminophen at the time of surgical wound closure. In preferred embodiments, the invention further comprises co-administering therapeutically effective doses of intravenous ibuprofen and acetaminophen about every 4 to about every 6 hours post-surgery, for at least about 8 doses, and in certain embodiments for up to about 120 hours (5 days) post-surgery.

Further embodiments of the invention entail administering an effective dose of intravenous ibuprofen prior to the start of surgery on the patient, thereafter performing surgery on the patient, administering an effective dose of acetaminophen at the time of surgical wound closure, and thereafter administering an effective dose of an opioid analgesic to the patient such that the patient experiences relief from pain associated with the surgery, the dose of opioid analgesic being lower than the dose of the opioid analgesic necessary to provide the same level of pain relief if the intravenous ibuprofen and/or acetaminophen is not administered. In preferred embodiments, the invention further comprises co-administering therapeutically effective doses of intravenous ibuprofen and acetaminophen about every 4 to about every 6 hours post-surgery, for at least about 8 doses, and in certain embodiments for up to about 120 hours (5 days) post-surgery. In certain embodiments, additional doses of opioid analgesic are administered to the patient at appropriate intervals, wherein the amount of opioid analgesic administered post-surgery is less than the amount of opioid analgesic administered to a patient(s) who is not administered intravenous ibuprofen/acetaminophen in accordance with the present invention.

In further embodiments, the administration of intravenous ibuprofen/acetaminophen in accordance with the present invention is opioid sparing when used for post-operative pain; provides a reduction in opioid side effects; provides a reduction in adverse events, provides a reduction in the need for anti-emetic medications, may allow patients to become ambulatory faster; and has an excellent safety profile, than if the intravenous ibuprofen/acetaminophen is not administered.

In further embodiments, the method comprises intravenously administering the effective dose of ibuprofen to a patient prior to the start of surgery at the onset of anesthesia.

Further embodiments of the invention are directed to a method of treating pain in human patients prior to the start of surgery via the administration of about 800 mg intravenous ibuprofen administered at the start of surgery, e.g., with the onset of anesthesia, and via the administration of about 1000 mg acetaminophen no later than at the completion of surgical wound closure. In preferred embodiments, the invention further comprises co-administering therapeutically effective doses of intravenous ibuprofen and acetaminophen about every 4 to about every 6 hours post-surgery, for at least about 8 doses, and in certain embodiments for up to about 120 hours (5 days) post-surgery.

In certain preferred embodiments, the patients are undergoing orthopedic surgery.

In additional embodiments, the invention is directed to a method of reducing surgical pain in human patients, comprising administering an effective dose (e.g., about 800 mg) of intravenous ibuprofen prior to the start of surgery on the patient, thereafter performing surgery on the patient, administering about 1000 mg acetaminophen no later than at the completion of surgical wound closure, and thereafter administering an effective dose of an opioid analgesic to the patient such that the patient experiences relief from pain associated with the surgery. In preferred embodiments, the method further comprises intravenously co-administering an 800 mg dose of ibuprofen and a 1000 mg dose of acetaminophen every six hours post-operatively to the patient. Preferably, the intravenous ibuprofen/acetaminophen and opioid therapy is continued until the patient no longer is suffering from post-operative pain.

In certain preferred embodiments, the human patients receiving the intravenous ibuprofen/acetaminophen therapy as described herein experience a significant reduction in pain as measured, e.g., by the VAS-AUC with movement for the post-operative period (hours 6-28 after completion of the surgical procedure).

In certain preferred embodiments, the human patients receiving the intravenous dose(s) of ibuprofen/acetaminophen as described herein require the administration of less opioid analgesic (e.g., morphine) than the dose of opioid typically required to provide an equivalent level of pain relief without the administration of intravenous ibuprofen/acetaminophen.

In certain preferred embodiments, the human patients receiving the intravenous dose(s) of ibuprofen/acetaminophen as described herein experience a significant reduction in pain as measured by the VAS at rest area under the curve and by the VRS for the post-operative period (hours 6-28 after completion of the surgical procedure).

In certain preferred embodiments, the human patient(s) experiences less pain via the intravenous administration of ibuprofen/acetaminohen as compared to typical patients undergoing the same procedure without the benefit of the intravenous administration of ibuprofen/acetaminophen.

In preferred embodiments, the human patient(s) receiving the intravenous dose(s) of ibuprofen/acetaminophen uses significantly less opioid analgesic. In certain preferred embodiments, the human patient(s) receiving intravenous ibuprofen/acetaminophen experiences about a 30% or greater reduction in mean opioid (e.g., morphine) consumption.

The invention is further directed to a safe and effective method for management of pain associated with orthopedic surgical procedures in a human patient(s), comprising intravenously administering a 800 mg dose of ibuprofen pre-surgically to the patient, and intravenously administering a 1000 mg dose of acetaminophen no later than at the completion of surgical wound closure in the patient. In preferred embodiments, the method further comprises intravenously administering an 800 mg dose of ibuprofen and a 1000 mg dose of acetaminophen every six hours post-operatively to the patient. In further embodiments, one or more opioid analgesics are administered to the human patient post-operatively, preferably, in an amount (of opioid analgesic) that is less than that typically required to control pain in human patients (due to the co-administration of intravenous ibuprofen/acetaminophen).

In preferred embodiments, patients receiving both ibuprofen and acetaminophen intravenously experience a significant reduction in pain as measured by the VAS with movement and at rest area under the curve for the first 24 hours, from 6 through 24 hours, and from 12 through 24 hours after surgery and a reduction in pain as measured by the VAS at rest area under the curve for the first 24 hours, from 6 through 24 hours, and from 12 through 24 hours.

### DETAILED DESCRIPTION OF THE INVENTION

NSAIDs are effective adjuncts to opioid analgesia for moderate to severe pain, resulting in pain relief and opioid dose sparing. NSAIDs alone could provide effective analgesia post-surgery when mild to moderate pain is expected. There is also evidence that, by avoiding or decreasing opioid use, NSAIDs can reduce the incidence of opioid associated adverse events.

In some embodiments of the present invention, a therapeutically effective dose of intravenous ibuprofen is administered to a patient for the treatment of post-operative pain. In certain preferred embodiments of the invention, the ibuprofen and acetaminophen are intravenously administered either immediately prior to the start of surgery, at the induction of anesthesia, during the surgical procedure, or at the time of surgical wound closure. In certain preferred embodiments, the dose of ibuprofen is administered at the induction of anesthesia. In certain preferred embodiments, the dose of acetaminophen is administered approximately at the induction of anesthesia. The dose of intravenous ibuprofen is deemed effective as measured by a reduction in the requirement for the narcotic (opioid) analgesic (e.g., morphine) post-surgery.

Patients who may be administered an intravenous ibuprofen dose in accordance with the present invention include those scheduled for elective single site surgery with anticipated need for post-operative I.V. morphine analgesia with anticipated use of >24 hours having adequate IV access. Examples of types of surgeries include orthopedic surgery (knee, hip or shoulder joint replacement or reconstruction), gynecologic including hysterectomy, major abdominal surgery including gall bladder, bowel or lower abdominal general investigative surgery (dual site bone graft orthopedic procedures qualify for inclusion). The dose of the intravenous ibuprofen can be from about 400 mg to about 800 mg, with dosing in certain embodiments occurring over a 30 minute interval of time every 6 hours. The post surgery dose of intravenous ibuprofen can be administered for up to 8 doses, up to about 120 hours (5 days) post-surgery, or can continue further on an every 6 hour or as needed basis.

Certain methods described herein comprise administering to a post surgical patient an intravenous pharmaceutical composition comprising ibuprofen. Intravenous pharmaceutical compositions of ibuprofen include any formulation suitable for administration to a patient via any intravenous method, including a bolus. In some embodiments the rate of infusion is such that the dose is administered over a period of about 30 minutes. In some embodiments the rate of infusion is such that the dose is administered over a period of less than 30 minutes. In some embodiments the rate of infusion is such that the dose is administered over a period of greater than 30 minutes.

Preferably, the pharmaceutical formulation of ibuprofen administered in accordance with the methods of the present invention is a formulation suitable for administration to a (e.g., human) patient via injection. Suitable injection methods include, in addition to intravenous injection, intraarterial infusion, intramuscular injection, transdermal injection, and subcutaneous injection.

In the present invention, it is further contemplated that a therapeutically effective dose of acetaminophen will also be administered to the patient along with the ibuprofen. In certain preferred embodiments of the invention, the ibuprofen and acetaminophen are intravenously administered either immediately prior to the start of surgery, at the induction of anesthesia, during the surgical procedure, or at the time of surgical wound closure. In certain preferred embodiments, the ibuprofen is intravenously administered at the induction of anesthesia.

In certain preferred embodiments, the dose of acetaminophen is administered at the time of surgical wound closure. Acetaminophen injection is indicated for the management of mild to moderate pain; the management of moderate to severe pain with adjunctive opioid analgesics; and the reduction of fever. Acetaminophen injection can be administered, e.g., as a 15 minute infusion or as a 30 minute infusion, and may be administered every 4 to 6 hours. The maximum recommended dose of acetaminophen on a daily basis to adults is 4000 mg. Accordingly, if the dosing of acetaminophen occurs every 4 hours, then in preferred embodiments the dose of acetaminophen administered is about 650 mg.

In certain preferred embodiments, the ibuprofen and the acetaminophen doses are administered intravenously at the same time post-surgery, at dosing intervals of every 4 to about every 6 hours.

Suitable carriers for intravenous administration include physiological saline or phosphate buffered saline (PBS), and solutions containing solubilizing agents, such as glucose, polyethylene glycol, and polypropylene glycol and mixtures thereof.

The formulation may include an aqueous vehicle. Aqueous vehicles include, by way of example and without limitation, Sodium Chloride Injection, Ringers Injection, Isotonic Dextrose Injection, Sterile Water Injection, Dextrose, and Lactated Ringers Injection. Nonaqueous parenteral vehicles include, by way of example and without limitation, fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include, by way of example and without limitation, sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include, by way of example and without limitation, ethyl alcohol, polyethylene glycol and propylene glycol for water miscible vehicles and sodium hydroxide, hydrochloric acid, citric acid or lactic acid for pH adjustment.

Typically a therapeutically effective dosage is formulated to contain a concentration of at least about 0.1% w/w up to about 90% w/w or more, such as more than 1% w/w of ibuprofen.

As used herein a “dosage regimen” refers to the protocol used to administer an intravenous pharmaceutical formulation comprising ibuprofen to a patient. In some embodiments the dosage regimen comprises a dose amount and dosing interval. In some embodiments the dosage regimen further comprises a dosing duration. As used herein “dosing duration” refers to the period of time over which a dose is administered. For example, if a volume of pharmaceutical composition comprising 400 mg of ibuprofen is administered over a dosing duration of 30 min and administration of a dose is initiated every 6 hours, then the dosage regimen is 400 mg, every six hours, administered over 30 minutes. In some embodiments the dosage duration is defined simply as 400 mg, every six hours.

In some embodiments described herein a dosage regimen for post surgical patients is defined as one that results in decreased usage of narcotic analgesic and/or decreased perception of pain and decreased side effects from use of a narcotic analgesic.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The invention is further directed to a method of treating at post surgical patient in need of pain relief comprising administering to the patient an intravenous pharmaceutical composition comprising ibuprofen at a dosage of (i) 400 mg ibuprofen or (ii) 800 mg ibuprofen. In certain preferred embodiments of this method, the dose of ibuprofen produces a decreased need for narcotic analgesic, decreased side effects from use of a narcotic analgesic and/or decreased perception of pain.

Several prescription and nonprescription brands of ibuprofen are approved for the treatment of fever, pain, and other indications. The recommended over the counter, single dose of oral ibuprofen to treat mild to moderate pain in adults is 400 mg every 4 to 6 hours. For chronic indications, such as rheumatoid arthritis and osteoarthritis, up to 3200 mg/day may be administered (300 mg qid, or 400/600/800 mg tid or qid).

The intravenous ibuprofen formulation of the present invention are the first and only intravenous formulation of ibuprofen available to treat mild to severe pain in adults and to reduce fever in children and adults. Oral ibuprofen has been available for more than 30 years and has an excellent record of efficacy and safety. The intravenous formulation is now commercially available in the U.S. 4-mL and 8-mL vials (each 100 mg/mL) for dilution in either saline or dextrose solution.

Examples of suitable IV ibuprofen treatments in accordance with the invention include the following: for simple adult dosing for pain: 800 mg every 6 hours. For simple adult dosing for fever: 400 mg every 4 to 6 hours. Critically ill patients may require higher doses for fever management. In addition, even in critically ill patients the dose may be adjusted up to 800 mg, not to exceed 3200 mg total daily dose. Intuitive pediatric fever dosing: 10-15 mg/kg up to 400 mg per single dose every 4 hours, consistent with pediatric oral dosing. The dose can be administered, e.g., via a 5-to-15-minute IV infusion.

As described herein, in clinical trials the intravenous ibuprofen when administered to human patients significantly reduced postoperative pain, measured at rest and with movement, and has a clinically significant opioid-sparing effect. There were significantly fewer patients in the IV ibuprofen treatment group with at least one morphine side effect compared with placebo patients. The intravenous ibuprofen was also highly effective in reducing fever in hospitalized patients. No bleeding or renal toxicity was reported in the clinical studies with intravenous ibuprofen.

As demonstrated, e.g., by the clinical study detailed in the appended example, the co-administration of ibuprofen and acetaminophen used in accordance with the invention provides a treatment for pain and/or fever using the intravenous route of administration, and is useful for the treatment of mild to severe pain and the reduction of fever in adults and children under 12 years of age. Co-administration of intravenous ibuprofen and acetaminophen used in accordance with the methods of the invention provides pain control, e.g., for abdominal and orthopedic surgical procedures; effective fever reduction, even in critically ill patients; is opioid sparing when used for post-operative pain; provides a reduction in opioid side effects; provides a reduction in adverse events, provides a reduction in the need for anti-emetic medications, may allow patients to become ambulatory faster; and has an excellent safety profile.

The co-administration of intravenous ibuprofen and acetaminophen in accordance with the present invention provide, e.g., the following benefits: speed relief of pain and/or fever to expedite release from a hospital or hospital-like setting; IV ibuprofen speeds relief to expedite hospital release, as demonstrated by a fast reduction of mild to severe pain and fever in adults and fever in children under 12 years of age; a reduction in pain at rest and with movement as measured by visual analog scores (VAS) following abdominal and orthopedic surgeries; a reduction in opioid side effects (nausea, vomiting, constipation); does not cause bleeding or renal concerns observed in clinical trials; and may provide an improvement in time to ambulation which may enable facilities to schedule additional procedures in the ambulatory setting.

Pain control in the postoperative setting and fever in critical care can be major concerns. Hospital/ambulatory care centers want to get patients on their feet and released sooner, but some pain control options have side effects and safety issues that can lengthen the hospital stay. Controlling pain is a challenge. Physicians are well aware of the JCAHO guidelines and are concerned about doing more to control pain. Dosing limitations of some agents make the task even more daunting, and opioid side effects make patients feel less like themselves. Physicians know and trust ibuprofen to control pain and fever. However, prior to the introduction of Caldolor®, ibuprofen was only available in an oral formulation and its use is limited in the hospital/ambulatory care setting.

Studies have shown that multimodal analgesic techniques can enhance recovery and patient outcome after ambulatory procedures, improving hospital throughput. For purposes of the present invention, multimodal refers to “balanced” analgesia. In other words, more than one modality of pain control can be used in order to obtain beneficial analgesic effect while reducing opioid-related side effects. Meta-analyses of NSAIDs (including ibuprofen) have shown robust effects on analgesia and/or opioid dose sparing, with corresponding reduction in opioid side effects.

The present invention is directed in part to a method for reducing pain in human patients undergoing surgical procedures with the administration of an opioid analgesic, comprising intravenously administering ibuprofen pre-surgically to the patient in an amount effective to significantly reduce post-operative pain in the patients, as measured by the VAS with movement and at rest area under the curve for the first 24 hours. In certain embodiments, the method further comprises the step of intravenously administering a dose of ibuprofen and acetaminophen every six hours post-operatively to the patient for at least 24 hours post-operatively. In certain embodiments, the method comprises intravenously administering an effective dose of ibuprofen and acetaminophen by approximately the completion of wound closure. The dose of ibuprofen is preferably about 800 mg and the dose of acetaminophen is preferably about 1000 mg.

A further aspect of the invention comprises administering one or more opioid analgesics to the human patients post-operatively. Preferably, the one or more opioid analgesics in an amount that is less than that typically required to control pain in human patients having undergone the same surgical procedure. In other words, intravenously administering the intravenous ibuprofen/acetaminophen to patients in a sufficient dose provides an opioid-sparing effect, enabling the reduction of the dose of opioid to the patients. In such embodiments, the dose of ibuprofen is about 800 mg. Preferably, the human patients receiving intravenous ibuprofen/acetaminophen experience at least a 20% reduction, or a 25% reduction, or at least a 30% reduction, or at least a 40% reduction in mean morphine consumption.

In further embodiments, the method further comprises intravenously administering the intravenous ibuprofen/acetaminophen in a sufficient dose to provide a reduction in side effects associated with the administration of opioid analgesics.

In yet further embodiments, the method further comprises intravenously administering the intravenous ibuprofen/acetaminophen in a sufficient dose to reduce pain scores in patients who are concurrently administered opioid analgesics, as compared to patients receiving opioid analgesics alone.

In certain preferred embodiments, the invention comprises intravenously administering the ibuprofen/acetaminophen in a sufficient dose such that the patients become ambulatory post-surgery at an earlier time point than if the intravenous ibuprofen is not administered.

In certain embodiments, the patient experiences a significant reduction in pain as measured by the VAS with movement and/or the VAS at rest area under the curve for time points within the first 24 hours after surgery. In certain preferred embodiments, the time points are from 0 through 24 hours, and from 6 through 24 hours, and from 12 through 24 hours after surgery.

In certain preferred embodiments of the invention, the ibuprofen and acetaminophen are intravenously administered every 6 hours post-surgery (e.g., starting with the onset of anesthesia, or wound closure).

The invention is further directed to a method of reducing surgical pain in human patients, comprising intravenously administering about 800 mg of ibuprofen prior to the start of surgery on the patients, administering about 1000 mg of acetaminophen by about the completion of wound closure, administering an effective dose of an opioid analgesic to the patient such that the patient experiences relief from pain associated with the surgery, the effective dose being an amount that is less than that typically required to control pain in human patients having undergone the same surgical procedure; and intravenously administering further effective doses of ibuprofen and acetaminophen every four to six hours post-operatively at least until 24 hours after surgery. In certain preferred embodiments, each dose of ibuprofen is about 800 mg and each dose of acetaminophen is about 1000 mg. In certain preferred embodiments, the doses of ibuprofen and acetaminophen are administered every six hours post-operatively to the patient until about 120 hours (five days) after surgery.

In preferred embodiments, the intravenous ibuprofen/acetaminophen combination provides an opioid-sparing effect, enabling the reduction of the dose of opioid to the patients. Preferably, the human patients receiving intravenous ibuprofen/acetaminophen combination experience at least a 20% reduction, or a 25% reduction, or at least a 30% reduction, or at least a 40% reduction in mean morphine consumption.

The invention is further directed to a method for improving the time to ambulation post-operatively in human patients undergoing orthopedic surgical procedures, comprising intravenously administering ibuprofen and acetaminophen every 6 hours intravenously post-surgery at least until 24 hours after surgery, and in certain embodiments until about 120 hours (5 days) post surgery. Preferably, this embodiment further comprises administering at least one opioid analgesic to the human patients post-operatively in an amount that is less than that typically required to control pain in human patients who have undergone the same surgical procedure.

The opioids are a group of drugs, both natural and synthetic, that are employed primarily as centrally-acting analgesics and are opium or morphine-like in their properties. The opioids include morphine and morphine-like homologs, including, e.g., the semisynthetic derivatives codeine (methylmorphine) and hydrocodone (dihydrocodeinone) among many other such derivatives. Morphine and related opioids exhibit agonist activity at central nervous system or CNS (referring to the brain and spinal cord) mu opioid receptors as well as showing affinity for the delta and kappa opioid receptors, to produce a range of effects including analgesia, drowsiness, changes in mood and mental clouding. In addition to potent analgesic effects, the morphine-related opioids may also cause a number of undesirable effects, including, for example, respiratory depression, nausea, vomiting, dizziness, mental clouding, dysphoria, pruritus, constipation, increased biliary tract pressure, urinary retention and hypotension.

Although morphine is typically used as the opioid analgesic during and/or post-surgery, one skilled in the art will recognize that other opioid analgesics can be used instead of part or all of the morphine. Opioid analgesics which may be used in accordance with the invention include, but are not limited to, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene, fentanyl, heroin, hydromorphone, hydroxypethidine, isomethadone, ketobemidone, levallorphan, levorphanol, levophen-acylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, tramadol, salts thereof, complexes thereof; mixtures of any of the foregoing, mixed mu-agonists/antagonists, mu-antagonist combinations salts or complexes thereof, and the like. In certain preferred embodiments, the opioid analgesic is a mu or kappa opioid agonist. In certain preferred embodiments, the opioid analgesic is morphine, dihydrocodeine, hydromorphone, hydrocodone, fentanyl, oxycodone, oxymorphone, salts thereof, and mixtures of any of the foregoing.

### DETAILED DESCRIPTION OF PREFERRED EMBODIMENTS

The following example represents specific embodiments of the foregoing discovery, and is not representative of the entire scope of the invention.

### Example 1

Example 1 is a randomized, single center, open-label trial to compare the safety and efficacy of intravenous ibuprofen (Caldolor®, commercially available from Cumberland Pharmaceuticals, Inc.) used alone versus Caldolor in combination with acetaminophen injection (Ofirmev®, commercially available from Cadance Pharmaceuticals, Inc.) in total knee or hip arthroplasty surgery in 78 adult patients between the ages of 18-65 undergoing knee or hip arthroplasty surgeries. The objective of the study was to compare the effectiveness of perioperative use of Caldolor (ibuprofen) injection alone and in combination with Ofirmev (acetaminophen) injection in total knee or hip arthroplasty orthopedic procedures.

Two groups of 39 patients were randomized to receive either 800 mg of Caldolor at the induction of anesthesia, followed by 800 mg of Caldolor every 6 hours until discharge for a total of up to 120 hours (5 days) (Group 1). Group 2 patients received 800 mg Caldolor at the induction of anesthesia and 1000 mg Ofirmev at the time of surgical wound closure, followed by 800 mg Caldolor plus 1000 mg Ofirmev every 6 hours until discharge for a total of up to 120 hours (5 days).

Effectiveness of Caldolor alone versus Caldolor and Ofirmev together was demonstrated by measuring patients' self-assessment of pain intensity using a visual analog scale (VAS; assessment completed at rest and with movement). Secondary end points were based on opioid requirements, patients' quality of recovery scale (QoR), length of hospital stay, length of PACU stay, PONV medication requirements, incidence of opioid-related side effects, and safety as determined by the incidence of treatment-emergent adverse events.

In the immediate post-operative period, there was no difference observed between the two groups. On day 3, Group 2 patients receiving 800 mg Caldolor plus 1000 mg Ofirmev exhibited lower VAS scores (p<0.002), compared to patients in Group 1 receiving only 800 mg Caldolor. There were no significant differences in QoR scores; Mean QoR (SD) was 177 for Group 1 (n=35) and 179.5 for Group 2 (n=39). Mean time to discharge from PACU for Group 1 was 85.6 minutes (SD=78.50), whereas time to discharge was 71.1 minutes (SD=78.97) for Group 2. Comparisons between study groups in terms of time to discharge from PACU were not statistically significant. In contrast, the incidence of adverse events (p<0.001), need for anti-emetic medications (p<0.001), and opioid consumption (p<0.001) was significantly reduced in Group 2 compared to Group 1. Table 1 provides a comparison of pain scores according to these study groups:

TABLE 1

| | Group 1 | | Group 2 | | 95% CI of | | |
|---|---|---|---|---|---|---|---|
| Pain Scores | n | Mean (SD) | n | Mean (SD) | difference | t-test (df) | p-value |
| Baseline | 35 | 6.3 (3.00) | 39 | 5.0 (3.32) | −0.99, 2.83 | 1.86 (72) | 0.067 |
| Day 1 | 35 | 6.8 (2.01) | 39 | 6.7 (2.21) | −0.87, 1.09 | 0.23 (72) | 0.822 |
| Day 2 | 35 | 6.5 (1.87) | 38 | 6.0 (2.08) | −0.41, 1.43 | 1.10 (71) | 0.274 |
| Day 3 | 33 | 6.7 (2.19) | 35 | 4.9 (2.49) | −0.71, 2.97 | 3.24 (66) | 0.002 |
| Day 4 | 28 | 5.8 (2.37) | 35 | 4.7 (2.38) | −0.10, 2.31 | 1.83 (61) | 0.071 |
| Day 5 | 25 | 5.8 (2.12) | 30 | 5.2 (2.36) | −0.62, 1.82 | 1.01 (53) | 0.607 |

Pain scores were slightly lower in subjects randomized into Group 2. There was statistical significance on Day 3 for the pain scores between both groups (6.7 in Group 1 vs. 4.9 in Group 2). Scores for Quality of Recovery were slightly higher in Group 2, which indicates a better recovery and time to discharge from PACU was lower in Group 2, these differences were not statistically significant. In addition, statistically significance was noted between both groups on incidence of adverse events, need for anti-emetic medications, and opioid consumption. This demonstrates that Group 2 had significant reduction in the opioid consumption, adverse events, and need for anti-emetic medications which may indicate significant clinical relevance.

Table 2 is a comparison between study groups for quality of recovery scores and time to discharge from PACU:

TABLE 2

| | Group 1 | | Group 2 | | Test | | | |
|---|---|---|---|---|---|---|---|---|
| | | Median | | Median | Statistics | | Effect | p- |
| | n(%) | (Range) | n(%) | (Range) | U | Z | size, r | value |
| Time to discharge | 35 (100) | 55 (5-321) | 39 (100) | 38 (1-342) | 558.0 | −1.35 | 0.15 | 0.178 |
| from PACU (mins) | | | | | | | | |
| Quality of | 35 (100) | 181 (133-197) | 39 (100) | 184 (128-198) | 586.5 | −1.04 | 0.12 | 0.298 |
| Recovery (QoR40) | | | | | | | | |

Table 3 is a comparison between study groups for duration of hospital stay:

TABLE 3

| | | | Chi-squared | |
|---|---|---|---|---|
| Hospital | Group 1 (N = 35) | Group 2 (N = 39) | value | |
| stay | n (%) | n (%) | (df) | p-value |
| 1 Day | 0 | 1 (2.6) | 6.959 (4) | 0.138 |
| 2 Days | 2 (5.7) | 3 (7.7) | | |
| 3 Days | 5 (14.3) | 0 | | |
| 4 Days | 3 (8.6) | 5 (12.8) | | |
| 5 Days | 25 (71.4) | 30 (76.9) | | |

Table 4 is a comparison between study groups for opioid requirement in PACU:

TABLE 4

| | Group 1 | | Group 2 | | | | |
|---|---|---|---|---|---|---|---|
| | | Mean | | Mean | 95% CI of | | |
| | n | (SD) | n | (SD) | difference | t-test (df) | p-value |
| Opioid | 35 | 24.6 (7.5) | 39 | 7.4 (3.4) | 14.35-19.92 | 12.39 (46.4) | <0.001 |
| requirement (mcg) | | | | | | | |

Table 5 is a comparison between study groups for incidence of adverse events reported during hospitalization:

TABLE 5

| Adverse | Group 1 (N = 35) | Group 2 (N = 39) | Chi-squared | |
|---|---|---|---|---|
| events | n (%) | n (%) | value (df) | p-value |
| With | 29 (82.9) | 1 (2.6) | 49.3 (1) | <0.001 |
| Without | 6 (17.1) | 38 (97.4) | | |

Table 6 is a comparison between study groups for need of anti-emetic medications during recovery time in PACU:

TABLE 6

| | | | Chi- | |
|---|---|---|---|---|
| Anti-emetic | Group 1 (N = 35) | Group 2 (N = 39) | squared | |
| medications | n (%) | n (%) | value (df) | p-value |
| Required | 19 (54.3) | 1 (2.6) | 25.0 (1) | <0.001 |
| Not required | 16 (45.7) | 38 (97.4) | | |

It was concluded that Caldolor plus Ofirmev, when coadministered, demonstrated significantly reduced adverse events, opioid consumption, and antiemetic medication requirements when compared to Caldolor administered alone. Furthermore, it was noted that on post-op Day 3 coadministration (combined administration) of Caldolor and Ofirmev provided improved pain scores in comparison to Caldolor alone suggesting a potential synergistic benefit.

### CONCLUSION

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method for reducing pain in a human patient undergoing a surgical procedure, comprising intravenously co-administering effective doses of ibuprofen and acetaminophen to the patient in an amount effective to significantly reduce post-operative pain in the patient, wherein the ibuprofen and acetaminophen are intravenously administered no later than at about the time of surgical wound closure, and thereafter co-administering therapeutically effective doses of intravenous ibuprofen and acetaminophen about every 4 to about every 6 hours post-surgery for at least about 8 doses.

2. The method of claim 1, wherein the intravenous ibuprofen and acetaminophen are administered about every 4 to about every 6 hours post-surgery for up to about 120 hours (5 days) post-surgery.

3. The method of claim 1, further comprising administering an opioid analgesic to the patient post-surgery.

4. The method of claim 3, wherein the administration of ibuprofen/acetaminophen delays the time to need for an opioid analgesic and/or reduces the amount of opioid analgesic administered to the patient post-surgery.

5. The method of claim 3, wherein the administration of intravenous ibuprofen/acetaminophen provides a reduction in side effects associated with the administration of opioid analgesics.

6. The method of claim 3, wherein the administration of intravenous ibuprofen/acetaminophen provides an effect selected from the group consisting of significantly reduced adverse events, significantly reduced opioid consumption, significantly reduced antiemetic medication requirements, and combinations of any of the foregoing, when compared to the ibuprofen administration without the acetaminophen administration.

7. The method of claim 3, wherein the administration of intravenous ibuprofen/acetaminophen reduces pain scores in the patient.

8. The method of claim 1, wherein the ibuprofen and the acetaminophen doses are administered intravenously at the same time post-surgery, at dosing intervals of every 4 to about every 6 hours.

9. The method of claim 1, wherein the ibuprofen is administered at about the induction of anesthesia.

10. The method of claim 8, wherein the acetaminophen is administered at about the time of surgical wound closure.

11. The method of claim 3, wherein the dose of opioid analgesic being lower than the dose of the opioid analgesic necessary to provide the same level of pain relief if the intravenous ibuprofen and acetaminophen are not administered.

12. The method of claim 3, wherein the co-administration of intravenous ibuprofen/acetaminophen provides an effect selected from the following: is opioid sparing as compared to the intravenous ibuprofen/acetaminophen not being administered; provides a reduction in opioid side effects as compared to the intravenous ibuprofen/acetaminophen not being administered; provides a reduction in adverse events as compared to the intravenous ibuprofen/acetaminophen not being administered; provides a reduction in the need for anti-emetic medications as compared to the intravenous ibuprofen/acetaminophen not being administered; allows the patient to become ambulatory faster as compared to the intravenous ibuprofen/acetaminophen is not administered; and combinations of any of the foregoing.

13. The method of claim 1, wherein the ibuprofen is administered to the patient prior to the start of surgery at about the onset of anesthesia.

14. The method of claim 1, wherein the dose of ibuprofen is about 800 mg.

15. The method of claim 1, wherein the dose of acetaminophen is about 1000 mg.

16. A method of treating surgical pain in a human patient, comprising administering about 800 mg ibuprofen intravenously at about the start of surgery, and administering about 1000 mg acetaminophen intravenously no later than at the completion of surgical wound closure.

17. The method of claim 16, further comprising co-administering therapeutically effective doses of intravenous ibuprofen and acetaminophen about every 4 to about every 6 hours post-surgery, for at least about 8 doses.

18. The method of claim 17, wherein the co-administration of intravenous ibuprofen and acetaminophen is for up to about 120 hours (5 days) post-surgery.

19. The method of claim 16, wherein the patient is undergoing orthopedic surgery.

20. The method of claim 16, further comprising administering therapeutic doses of an opioid analgesic post-surgery in order to further treat pain in the patient.

21. The method of claim 16, wherein the ibuprofen is first administered at about the induction of anesthesia.

22. The method of claim 18, wherein the co-administered doses are about 800 mg ibuprofen and about 1000 mg acetaminophen.

23. The method of claim 17, wherein the human patient receiving intravenous ibuprofen/acetaminophen experiences about a 30% or greater reduction in mean opioid consumption as compared to a patient who does not receive the coadministration of intravenous ibuprofen/acetaminophen.

24. The method of claim 1, wherein the patient receiving both ibuprofen and acetaminophen intravenously experiences a significant reduction in pain as measured by the VAS with movement and at rest area under the curve for the first 24 hours, from 6 through 24 hours, and from 12 through 24 hours after surgery and a reduction in pain as measured by the VAS at rest area under the curve for the first 24 hours, from 6 through 24 hours, and from 12 through 24 hours.
